(19) United States
(12) Patent Application Publication
(10) Pub. No.: US 2023/0355191 A1
(43) Pub. Date: Nov. 9, 2023

(54) System and Method for Presentation of Anatomical Orientation of 3D Reconstruction

(71) Applicant: GE Precision Healthcare LLC, Wauwatosa, WI (US)

(72) Inventors: Liliane Ramus, Versailles (FR); Régis Vaillant, Villebon sur Yvette (FR); Benjamin Wimille, Versailles (FR)

(21) Appl. No.: 17/737,143

(22) Filed: May 5, 2022

(51) Int. Cl. A61B 6/12; A61B 6/00; G06T 7/73

(52) U.S. Cl. CPC A61B 6/12; A61B 6/463; A61B 6/4085; A61B 6/4441; A61B 6/5205; G06T 7/73; A61M 2025/1079

(57) ABSTRACT

An x-ray imaging system and method of operation includes a gantry having an x-ray source and an x-ray detector alignable with the x-ray source, along with an image processing system operably connected to the gantry and including a processing unit for processing the x-ray image data from the detector to form x-ray images. The processing unit is configured to determine a position of at least two radiopaque markers located on an object within the anatomy of a patient, where the at least two radiopaque markers identify a proximal end of the object and a distal end of the object. The processing unit can then form one or more x-ray images of the object and the anatomy, and present at least two different indicators representing the radiopaque markers within the one or more x-ray images on the display to provide anatomical orientation information regarding the object within the patient anatomy.

### FIELD OF THE DISCLOSURE

This disclosure relates generally to systems and methods of stent imaging and display. More specifically, the present disclosure relates to providing information to a viewer regarding the anatomical orientation of a stent in conjunction with a motion-corrected image of a stent within the anatomy.

### BACKGROUND OF THE DISCLOSURE

A stent is a metal coil or mesh tube that can be placed within a lumen, which can be a blood vessel, in order to provide support and/or to keep the lumen open. Stents may be implemented to treat a variety of medical conditions, for example, an aneurysm which is the dilation of a blood vessel resulting in stretching of the vessel wall, or a stenosis which is a partial or a total occlusion of a blood vessel.

A conventional procedure for placing a stent includes the following sequence of steps. A guidewire is initially inserted at the point of entry, which is usually a small percutaneous incision in the arm or groin, and is then guided through one or more blood vessels to the target site (e.g., a site defined at or near the aneurysm or the stenosis). Thereafter a hollow generally cylindrical catheter is slipped over the guidewire and directed to the target site by following the guidewire. The stent is generally compressed or compacted in order to facilitate its navigation through the catheter to the target site. Thereafter, the stent is expanded, such as through the use of an expandable balloon or other similar device located on the catheter, to support a localized region of the vessel wall and/or to keep the vessel open.

The stent must be precisely positioned at a predetermined location within the blood vessel (e.g., at the dilation or occlusion) in order to most effectively treat the underlying medical condition. The stent is maneuvered by sliding along the guidewire. Stent placement precision is related to the accuracy with which it is placed with respect to the target site. Fluoroscopic or other radiographic imaging can be used to track and navigate the guidewire and other tools (e.g. catheter, balloon, stent) to the deployment location.

After the stent is deployed in the vessel, it is desirable to confirm proper stent deployment before completion of the procedure. If any errors are identified with regard to the deployment of the stent, the clinician can take corrective action, e.g. re-inflate the balloon. However, most often the deployed stent is barely visible in X-ray images and must be enhanced with image processing techniques. One exemplary manner of obtaining the images of the stent is through utilizing cone beam computed tomography (CBCT) where a conical beam of x-rays from the CBCT imaging device, such as a C-arm fluoroscopic device, is directed towards the object of interest within the patient. The multiple x-ray images or projections obtained by the CBCT device at different angles of the object of interest, e.g. the stent, are then processed on a computer using reconstruction algorithms to produce tomographic (cross-sectional) images of the object within the body.

One key limit of the CBCT technology is the capability of reconstructing moving objects since the acquisition speed for the CBCT imaging device is limited, particularly with regard to addressing motion of the object of interest and/or patient while obtaining the x-ray images. More specifically, at least one of the x-ray source and the x-ray detector of the CBCT device is carried by a C-arm. Further, the rotational speed of the C-arm is much slower than the rotational speed of a conventional fan beam CT scanning device, which can overcome the challenge of movement of the patient and/or object of interest by acquiring the necessary projections in a very short time such that moving objects can be considered static or non-moving. With the slower rotational speed of the C-arm of the CBCT imaging device, the projections obtained by the CBCT device must be motion corrected to provide an accurate reconstruction image of the object, i.e., stent, within the patient.

Typical stent enhancement techniques are performed by combining several images of the stent after motion compensation. The small size of the stent and the component struts present one challenge, but a greater challenge is compensating for the movement of and within the patient.

One example of a motion compensation technique relies on detecting, across several images, radiopaque markers that are attached to the expandable delivery balloon located on the catheter along with the stent. The delivery balloon is held in position relative to the deployed stent and the markers detected in each of the projections are used to estimate and compensate for the patient and stent motion. The most common application of this approach is for the reconstruction of an artery section with or without a stent where the two markers are placed in the artery by sliding the balloon including the markers along a guidewire. This method provides adequate reconstruction of moving objects such as the stent in a 3D image or volume, but the reconstruction is limited to the volume which follows the motion described by the markers. The user is then presented with the difficulty of determining the position and the orientation of this limited volume with respect to the full patient anatomy in order to discern the proper anatomical orientation of the stent, as the reconstructed 3D image/volume is produced in a geometry defined by the two markers. From an anatomical point of view, one marker is considered as proximal i.e., closer to the ostia of the artery and the other is considered as distal i.e., on the other side. but from an algorithm point of view, the two markers are strictly equivalent and thus provide no indication of anatomical orientation within the 3D image.

Another example of an exemplary method of stent enhancement in medical images includes obtaining a plurality of medical images such as that disclosed in U.S. Pat. No. 10,467,786 entitled Systems And Methods Of Stent Image Enhancement (the '786 patent), the entirety of which is expressly incorporated herein by reference for all purposes. In this process, a plurality of medical images/projections are obtained as temporally successive images of a stented vessel. A centerline of the stented vessel in each medical image of the plurality of medical images is obtained. A deformation field across the plurality of images is estimated based at least in part upon the obtained centerline in each of the images. The plurality of medical images are then registered to a common reference image to correct for the motion. The registered images are temporally integrated to obtain a contrast-enhanced image of the stent and local patient anatomy.

However, in the various processes of obtaining the motion correction for the image of the stent, the representation of the anatomy around the stent is lost, such that it is difficult to determine the orientation of the stent within the anatomy motion-compensated image, i.e., which end of the vessel and/or stent is the proximal end and which end is the distal end. For an enhanced 2D image of the stent, this loss of the representation of the anatomy surrounding the stent is not overly problematic because the orientation of the stent in the enhanced 2D image is the same as that of the X-ray image sequence used to compute the stent enhanced image. Conversely, the loss of the surrounding anatomy is more important in 3D because the orientation of the stent in the 3D rotational acquisition is changing along the image sequence. Consequently, the inability to readily determine the proximal and distal ends of the vessel and/or stent in the reconstructed, motion-corrected images presents a clinical risk in that the error in the interpretation of proximal/distal side of a 3D image or volume may lead to an erroneous intervention for the patient.

Prior art systems and methods have been devised to fix the problem such as displaying the image chain angulation which corresponds to a 3D view of the reconstructed objects or requiring the user to mark the proximal marker in one of the projections and then reconstructing the 3D image/volume in the geometry indicated by the user, i.e. with the proximal marker on the top. However, these corrective measures require significant additional steps and user interaction, and, particularly with regard to any user interaction and errors therein, may not adequately provide the anatomical orientation information necessary for the proper determination of the orientation of the stent within the patient anatomy.

In addition, to address this issue intravascular imaging can be employed which also provides 3D view of the stent within the anatomy. In intravascular imaging the proximal/distal sides are known by design because the intravascular imaging catheter positioned within the anatomy first acquires images of the distal side of the stent and is then pulled back within the anatomy, i.e., along the artery, towards the ostia to acquire images of the proximal side. However, the need for the separate imaging catheter and the procedure for the positioning and movement of the imaging catheter relative to the stent to obtain the images adds significant complexity and time to the determination of the orientation of the stent within the anatomy within the computed images.

As a result, it is desirable to develop a system and method that provides an indication to the clinician of the orientation of the stent within the anatomy in conjunction with a motion-corrected 3D image of the stent.

### SUMMARY OF THE DISCLOSURE

According to one aspect of an exemplary embodiment of the disclosure, an x-ray imaging system including an x-ray source and an x-ray detector alignable with the x-ray source, an image processing system operably connected to the x-ray source and x-ray detector to generate x-ray image data, the image processing system including a processing unit for processing the x-ray image data from the detector to form x-ray images, a database operably connected to the processing unit and storing instructions for operation of the processing unit, and a display operably connected to the image processing system for presenting the x-ray images to a user, wherein the processing unit is configured to determine a position of at least two radiopaque markers located on an object within the anatomy of a patient, the at least two radiopaque markers identifying a proximal end of the object and a distal end of the object, to form one or more x-ray images of the object and the anatomy, and to present at least two different indicators representing the radiopaque markers within the one or more x-ray images on the display.

According to still another aspect of an exemplary embodiment of the present disclosure, a method for providing anatomical orientation information in conjunction with images provided by an x-ray imaging system includes the steps of providing an x-ray imaging system having a gantry including an x-ray source, and an x-ray detector alignable with the x-ray source, an image processing system operably connected to the gantry to control the operation the x-ray source and x-ray detector to generate x-ray image data, the image processing system including a processing unit for processing the x-ray image data from the detector to form x-ray images, a database operably connected to the processing unit and storing instructions for operation of the processing unit, a display operably connected to the image processing system for presenting information to a user, and a user interface operably connected to the image processing system to enable user input to the image processing system, positioning an object including at least two radiopaque markers within the anatomy of a patient, the at least two radiopaque markers identifying a proximal end of the object and a distal end of the object, operating the x-ray source to obtain x-ray image data of the object and the anatomy, determining a position of the at least two radiopaque markers within the anatomy from the x-ray image data, forming one or more x-ray images of the object and the anatomy from the x-ray image data and presenting at least two different indicators representing the radiopaque markers on the one or more x-ray images.

These and other exemplary aspects, features and advantages of the invention will be made apparent from the following detailed description taken together with the drawing figures.

### BRIEF DESCRIPTION OF THE DRAWINGS

The drawings illustrate the best mode currently contemplated of practicing the present invention.

In the drawings:

FIG. 1 is a block schematic diagram of an exemplary imaging system according to an exemplary embodiment of the disclosure.

FIG. 2 is a schematic diagram of an exemplary balloon catheter structure according to an exemplary embodiment of the disclosure.

FIG. 3 is a schematic view of the display of 3D reconstructed images of the stent including indications illustrating the anatomical orientation of markers on the stent within the patient anatomy according to an exemplary embodiment of the disclosure.

FIG. 4 is a schematic view of the display of a 2D projection image of the patient anatomy including indicators of illustrating the anatomical orientation of markers on the stent also shown in the 3D images relative to the patient anatomy according to an exemplary embodiment of the disclosure.

### DETAILED DESCRIPTION OF THE DRAWINGS

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

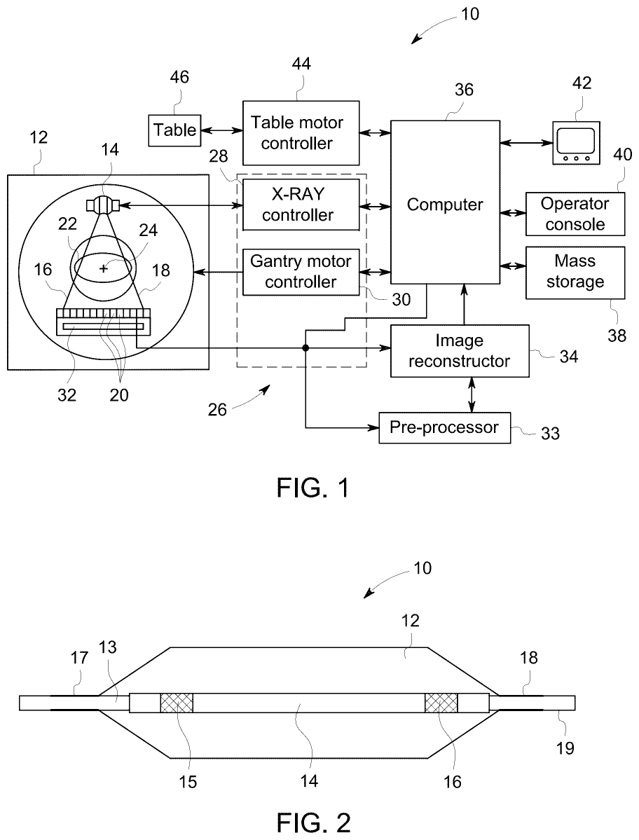

The following description relates to various embodiments of medical imaging systems any of which may be suitably used in the planning, provision, and evaluation of stent placement. FIG. 1 depicts an exemplary embodiment of an imaging system 10 which may be used to obtain the medical images as described herein of the patient. Though an x-ray system is described by way of example, it should be understood that the present techniques may also be useful when applied to images acquired using other imaging modalities, including, but not limited to fluoroscopy and C-arm angiography and/or vascular imaging procedures. The present discussion of is provided as an example of one suitable application.

Referring to FIG. 1, an exemplary embodiment of the system 10, such as that disclosed in U.S. Pat. No. 10,467,786, entitled Systems and Methods of Stent Image Enhancement, the entirety of which is hereby expressly incorporated by reference for all purposes, may be utilized to obtain x-ray images or projections of the anatomy and/or an object disposed within or as part of the anatomy of a patient.

The imaging system 10 is shown as including a gantry 12. Gantry may be a substantially C-shaped or semi-circular gantry, or C-arm gantry. The gantry 12 movably supports a source 14 and a detector 18 mounted opposite to each other on opposed ends. Further, a subject 22 is positioned between the source 14 and the detector 18.

Gantry 12 includes an x-ray source 14 that projects a beam of x-rays 16 toward detector array 18. The gantry 12 exemplarily includes a lower end 13 that is positioned below a subject 22, such as a patient, and an upper end 15 that is positioned above the subject 22. The x-rays pass through the subject 22 and any object 24 positioned within the subject/patient anatomy 22 to generate attenuated x-rays. As depicted in FIG. 1, the x-ray source 14 may be secured to the one end and the x-ray detector 18 secured to the other end. Each detector element 20 is exemplarily, but not limited to a cadmium telluride (CdTe) detector element, which produces an electrical signal that represents an intensity of the attenuated x-rays.

During a scan to acquire image data, gantry 12 and/or components mounted on gantry 12 are movable relative to the subject 22 and/or a table 46. The table 46 may include a scanning surface on which the subject 22 may be positioned. For example, during an acquisition of image data, the gantry 12 is movable to change a position and/or orientation of the source 14 and/or detector 18 relative to the patient. In an exemplary embodiment, the gantry 12 may move the source 14 and the detector 18 in a rotational scanning path 23 that moves around the patient/subject 22. It will be recognized that other forms of image data acquisition may utilize other forms of scanning paths, which may include, but are not limited to rotation or tilt of the gantry 12. It will be recognized that in other exemplary imaging systems within the present disclosure, one of the source or detector may remain in a fixed position while the other of the source or detector is movable with respect to the patient. In still other exemplary embodiments as disclosed herein, the table, which is configured to support the patient, is further movable to achieve a desired image acquisition.

Movement of the gantry 12 and an operation of x-ray source 14 are governed by an imaging controller 26 of imaging system 10. Imaging controller 26 includes an x-ray controller 28 that provides power and timing signals to x-ray source 14. The x-ray controller 28 may further provide operational and/or control signals to the adjustable collimator 25 to shape the beam of x-rays from the source 14 in accordance with the imaging procedure to be performed. In some embodiments, the x-ray beam may be shaped (collimated) as a cone beam, such as where the imaging system 10 is formed as a C-arm computed tomography (CT) system and/or operated as a cone beam computed tomography (CBCT) system.

The imaging controller 26 further includes a gantry motor controller 30 that controls a motion, speed, and position of gantry 12. In some embodiments, gantry motor controller 30 may control a tilt angle of gantry 12. The gantry motor controller 30 may further operate to control a movable joint (not shown) between the detector 18 and the gantry 12. The gantry motor controller 30 may further operate to control a movable joint (not shown) exemplarily between the source 14 and the gantry 12. The table motor controller 44 is operably connected to the table 46 through a table motor (not shown). The table motor is operable, under control signals from the table motor controller 44, to translate, rotate, and/or tilt the table 46 in a plurality of degrees of freedom of movement. In an embodiment, the table motor is operable to move the table 46 in three degrees of freedom, (e.g. horizontal, vertical, and depth translation) while in another embodiment, rotational degrees of freedom of movement (e.g. pitch, yaw, and roll) may be available. It will be recognized that the table motor may include one or more mechanical or electromechanical systems to carry out these movements of the table 46, including but not limited to tack and opinion, screw, or chain driven actuators.

The x-ray source 14 and the x-ray detector 18 may be moved in a rotational direction/pattern 23 so as to obtain a series of angular position scans of the subject 22 during which x-ray data is collected by the x-ray detector 18. The rotational scanning procedure generates a quantitative image data set from a plurality of scan images acquired at the various angular positions around the patient 22, wherein the x-ray source 14 and the detector 18 are disposed in alignment with one another at each angular position.

The rotational scanning motion/path 23 is produced by coordination between the motion control of the gantry 12, x-ray source 14, and the x-ray detector 18 by the gantry motor controller 30 as well as control of the table 46 by the table motor controller 44 which operates the table 46 through the table motor. During operation, the x-ray source 14 produces a cone beam 16, though the x-ray source 14 may also be configured to output a pencil beam of x-rays (not shown), a fan beam of x-rays, or other configurations.

A data acquisition system (DAS) 32 in the imaging controller 26, samples and digitizes the data from detector elements 20 and converts the data to sampled and digitized data for subsequent processing. In some embodiments, DAS 32 may be positioned adjacent to detector array 18 on gantry 12. Pre-processor 33 receives the sampled and digitized data from DAS 32 to pre-process the sampled and digitized data. In one embodiment, pre-processing includes, but is not limited to, an offset correction, a primary speed correction, a reference channel correction, an air-calibration, and/or applying a negative logarithmic operation. As used herein, the term processor is not limited to just those integrated circuits referred to in the art as a processor, but broadly refers to a controller, a microcontroller, a microcomputer, a programmable logic controller, an application specific integrated circuit, and any other programmable circuit, and these terms are used interchangeably herein. Pre-processor 33 pre-processes the sampled and digitized data to generate pre-processed data.

An image processing system 35 receives the pre-processed data from pre-processor 33 and performs image analysis, including that of motion correction, 2D and/or 3D image reconstruction, through one or more image processing operations. The pre-processed data may be processed and displayed in real time though operations of a image reconstructor 34 and/or a processing unit/computer 36 forming the image processing system 35. The processing unit 36 exemplarily operates to store the reconstructed image in a mass storage device 38, where the mass storage device 38 may include, as non-limiting examples, a hard disk drive, a floppy disk drive, a compact disk-read/write (CD-R/W) drive, a Digital Versatile Disc (DVD) drive, a flash drive, and/or a solid-state storage device. As used herein, the term computer is not limited to just those integrated circuits referred to in the art as a computer, but broadly refers to a processor, a microcontroller, a microcomputer, a programmable logic controller, an application specific integrated circuit, and any other programmable circuit, and these terms are used interchangeably herein. It will be recognized that any one or more of the processors and/or controllers as described herein may be performed by, or in conjunction with the processing unit 36, for example through the execution of computer readable code stored upon a computer readable medium accessible and executable by the processing unit 36. For example, the computer/processing unit 36 may include a processor configured to execute machine readable instructions stored in the mass storage device 38, which can be non-transitory memory. Processor unit/computer 36 may be single core or multi-core, and the programs executed thereon may be configured for parallel or distributed processing. In some embodiments, the processing unit 36 may optionally include individual components that are distributed throughout two or more devices, which may be remotely located and/or configured for coordinated processing. In some embodiments, one or more aspects of the processing unit 36 may be virtualized and executed by remotely-accessible networked computing devices configured in a cloud computing configuration. According to other embodiments, the processing unit/computer 36 may include other electronic components capable of carrying out processing functions, such as a digital signal processor, a field-programmable gate array (FPGA), or a graphic board. According to other embodiments, the processing unit/computer 36 may include multiple electronic components capable of carrying out processing functions. For example, the processing unit/computer 36 may include two or more electronic components selected from a list of electronic components including: a central processor, a digital signal processor, a field-programmable gate array, and a graphic board. In still further embodiments the processing unit/computer 36 may be configured as a graphical processing unit (GPU) including parallel computing architecture and parallel processing capabilities.

Processing unit 36 also receives commands and scanning parameters from a user, such as an operator, via a console 40 that includes a user interface device, such as a keyboard, mouse, voice-activated controller, touchscreen or any other suitable input apparatus. An associated display 42 allows a user, such as an operator, to observe the image from processing unit 36. The commands and scanning parameters are used by processing unit 36 to provide control signals and information the imaging controller 26, including the DAS 32, x-ray controller 28, and gantry motor controller 30. In addition, processing unit 36 may operate a table motor controller 44 exemplarily of the imaging controller 26 which controls a movable subject support, which is exemplarily a motorized table 46, to position subject 22 within gantry 12. Particularly, table motor controller 44 adjusts table 46 to move portions of subject 22.

Looking now at FIG. 2, the object 24 can take the form of an interventional device 101, such as a catheter 100 according to an exemplary embodiment of the invention, In the illustrated exemplary embodiment, the catheter 100 has a cylindrical inner shaft 103 through which a guide wire 110 can extend when the catheter 100 is inserted into the body of the patient 22, and a balloon 102 disposed around the inner shaft 103, which is illustrated in a dilated state for the deployment of the stent 200 positioned thereon within the blood vessel or other structure of the patient 22. A polymer carrier in the form of a polymer carrier strip 104 is fastened to the outer periphery of the inner shaft 103 within the balloon 102. A distal radiopaque marker 105 and a proximal radiopaque marker 106 are arranged on the polymer carrier strip 104 at a precisely defined distance from one another. The balloon 102 is connected in a distal region 107 to an outer shaft 109 and in a proximal region 108 to the inner shaft 103, in each case in a fluid-tight manner The outer shaft 109 has the function of directing a fluid, i.e., a gas or liquid, through the outer shaft 109 and into the balloon 102 for inflating and deflating the balloon 102.

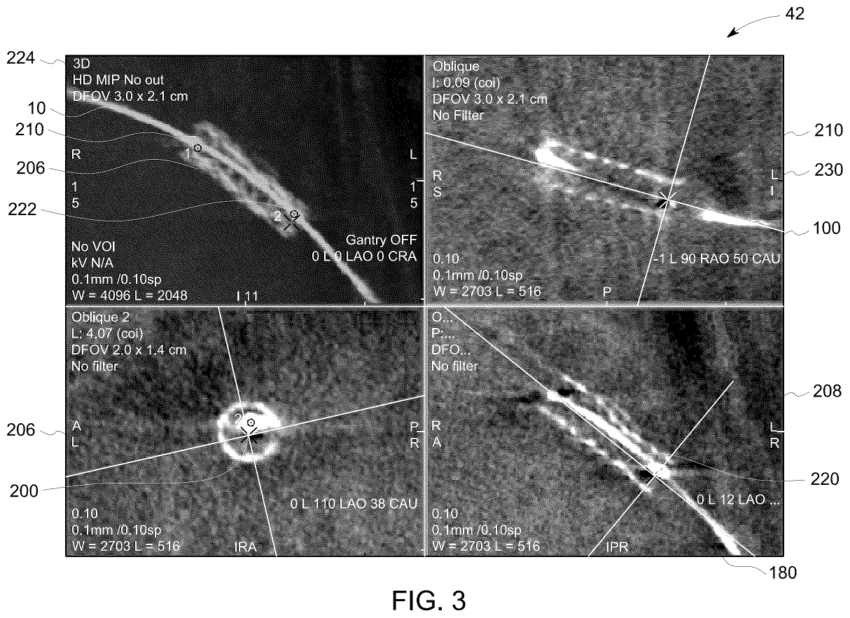

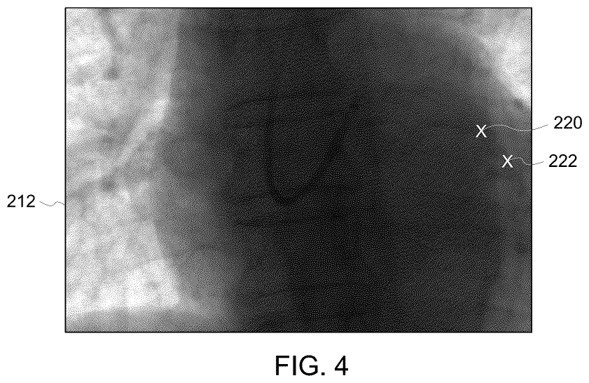

Referring now to FIGS. 3 and 4, the image data is employed by the image processor 34, such as in a computed tomography reconstruction, to produce one or more motion-corrected 3D volumetric images 204-210 of the stent 200, which are illustrated or presented on the display 42. The 3D images 204-210 provide viewable representations of the stent 200 within the immediately surrounding patient anatomy along selected or predetermined view axes or angles such that the deployment of the stent 200 can be verified in association with the patient anatomy. In the illustrated exemplary embodiment, the display 42 can present the images 204-210 simultaneously, with each image 204-210 present in a separate area or quadrant of the display 42. However, as shown in each of the images 204-210, while the stent 200 is discernable within the anatomy, the anatomical orientation of the stent 200 cannot readily or easily be determined from any of the images 204-210.

In one exemplary embodiment of the disclosure, the images 204-210 produced by the image reconstructor 34 and/or computer 36 include a determination of the locations of the markers 105, 106 within the image data. As the markers 105, 106 are able to be readily detected in the image data by the reconstructor 34 and/or the computer 36, the positions of the markers 105, 106 can be determined with respect to the stent 200 in each of the 3D images 204-210. After this determination, the locations of the markers 105, 106 can be represented in each of the motion-corrected 3D images 204-210 by a suitable and readily differentiable indicator 220, 222 such as various types of differentiable symbols superimposed over the respective image 204-210 in which the position of the marker 105, 106 is visible. As some images 204-210 will not show all or any of the markers 105, 106 due to the orientation of the particular image 204-210, the presence and/or position of the indicators 220, 222 will vary between images 204-210, as shown in FIG. 3.

Further, because the positions of the markers 105, 106 are known in the image data used to form the images 204-210, in addition to the processing of the image data to obtain the images 204-210, as best shown in FIG. 4, the image reconstructor 34 and/or the computer 36 are also operated utilizing suitable algorithms, instructions and/or operators to present an anatomical orientation reference image 212 in conjunction with the 3D images 204-210. The anatomical orientation reference image 212 provides an anatomical reference on the display to enable the user of the system 10 to properly orient the position of the stent 200 within the patient anatomy. The anatomical orientation reference image 212 is presented as a 2D image of a wider anatomical region of the patient 22 that includes the area shown in images 204-210. While any suitable 2D image collected in the CBCT acquisition can be employed as the reference image 212, such as a side view, in the exemplary illustrated embodiment the reference image 212 is a 2D front view of the entire imaged volume. Within the larger region contained within the reference image 212, the same indicators 220, 222 provided in the images 204-210 to indicate the location of the markers 105, 106 are presented to show the location of the stent 200 within the larger anatomical region in the reference image 212. The 3D images 204-210 can be subsequently presented with the reference image or view 212 on the display 42, such as with the reference image 212 in place of one of the 3D images 204-210 in a quadrant of the display 42, where the reference image 212 enables the identification of the proximal side and distal side of the artery, and in which the catheter tip and/or the guidewire tip are visible.

In addition, to more readily enable the user to determine the orientation of the stent 200 within the reference image 212 and within the 3D images 204-210, the image reconstructor 34 and/or computer 36 are operated to provide different shapes, colors and/or other differentiable features or attributes to the indicators 220, 222 represented in each of the 3D images 204-210 and the reference image 212, e.g., a first color symbol (e.g., blue) for the indicator 220 representing the proximal side of the stent 200, and a second color symbol (e.g., green) that is readily discernable from the first color for the indicator 222 representing the distal side of the stent 200. With the different shapes, colors, combinations thereof, etc. provided to the indicators 220, 222, the orientation presented in the reference image 212 can be easily translated by the user into each of the 3D images 204-210 in order to provide the proper anatomical orientation to the position of the stent 200 within the 3D images 204-210. With the proper orientation, the user can then quickly and correctly determine what portion of the stent 200 may need adjustment with regard to the surrounding patient anatomy, and then proceed to make the proper adjustment to the engagement and/or position of the stent 200 within the blood vessel or other anatomical lumen.

In other exemplary embodiments of the present disclosure, the markers 105, 106 can be applied directly to or formed on or within the stent 200, and/or to other portions of the catheter 100, such as the tip (not shown) of the guide wire, with precise known locations in order to provide additional or substitute points of orientation for indicators 220, 222 to be represented in the 3D images 204-210 and the reference image 212.

In still other exemplary embodiments of the present disclosure, the display 42 can present the 3D images 204-210 optionally without the reference image 212, but with the indicators 220, 222 shown in the 3D images 204-210 to provide a sufficient anatomical orientation of the position of the stent 200 within the patient anatomy.

In still other exemplary embodiments, more than two markers 105, 106 can be present on the stent 200, such that more than two indicators 220, 22 are present in the 3D images 204-210 and the reference image 212. Additionally, the markers 105, 106 can be present on one or more interventional devices 101 other than a stent 200, such as valves, occluders, left atrium appending closing devices, etc., to illustrate the orientations of these devices 101 within in the 3D images 204-210 and the reference image 212, alone or in combination with a stent 200 and/or one another. In essence, the disclosed system and method can be used to provide indications 220, 222 in 3D and 2D images providing an easily determinable anatomical orientation of any device(s) 101 imaged with x-ray imaging equipment/system 10 while positioned/placed in a moving anatomy that is being imaged and having at least two radio opaque markers on and/or moving with the device(s) 101, where the two radio opaque markers may be used for motion compensation. The method and system of the present disclosure can also be employed to reconstruct and label local vessel anatomy and/or features, such as calcifications, by simply placing the device 101, e.g., a balloon 102 with its two radiopaque markers 105, 106, inside the artery at the desirable location where the markers 105, 106 can be used as reference points for the location of the features in the 3D and 2D images constructed by the system 10.

It is understood that the aforementioned compositions, apparatuses and methods of this disclosure are not limited to the particular embodiments and methodology, as these may vary. It is also understood that the terminology used herein is for the purpose of describing particular exemplary embodiments only, and is not intended to limit the scope of the present disclosure which will be limited only by the appended claims.

We claim:

1. A method for providing anatomical orientation information in conjunction with images provided by an x-ray imaging system, the method comprising the steps of:
a. providing an x-ray imaging system comprising:
i. an x-ray source, and an x-ray detector alignable with the x-ray source;
ii. an image processing system operably connected to the x-ray source and x-ray detector to generate x-ray image data, the image processing system including a processing unit for processing the x-ray image data from the detector to form x-ray images, a database operably connected to the processing unit and storing instructions for operation of the processing unit, and a display operably connected to the image processing system for presenting information to a user; and
b. positioning an object including at least two radiopaque markers within the anatomy of a patient, the at least two radiopaque markers identifying a proximal end of the object and a distal end of the object;
c. operating the x-ray source to obtain x-ray image data of the object and the anatomy;
d. determining a position of the at least two radiopaque markers within the anatomy from the x-ray image data;
e. forming one or more x-ray images of the object and the anatomy from the x-ray image data; and
f. presenting at least two different indicators representing the radiopaque markers on the one or more x-ray images.

2. The method of claim 1, wherein the step of presenting the at least two different indicators comprises:
a. providing a first indicator having a first differentiable feature; and
b. providing a second indicator having a second differentiable feature.

3. The method of claim 1, wherein the step of forming one or more x-ray images comprises:
a. forming at least one 3D image of the object and the anatomy; and
b. forming a 2D image of the object and the anatomy.

4. The method of claim 3, wherein the step of presenting the at least two different indicators comprises:
a. presenting the at least two different indicators on the at least one 3D image; and
b. presenting the at least two different indicators on the 2D image.

5. The method of claim 3 wherein the step of forming the 2D image comprises forming a 2D frontal view of the object and the anatomy.

6. The method of claim 3, wherein step of presenting at least two different indicators representing the radiopaque markers on the one or more x-ray images comprises:
a. presenting the at least one 3D image with the at least two different indicators on the display; and
b. presenting the 2D image with the two different indicators on the display adjacent the at least one 3D image.

7. The method of claim 3, wherein the step of forming the at least one 3D image comprises forming at least one motion-corrected 3D image of the object and the anatomy.

8. The method of claim 1, wherein the x-ray imaging system is a C-arm system.

9. The method of claim 7, wherein the x-ray imaging system is a CBCT system.

10. The method of claim 1, wherein the object is a catheter.

11. The method of claim 9, wherein the object is a balloon catheter.

12. The method of claim 1, wherein the object is a stent.

13. An x-ray imaging system comprising:
a. a gantry including an x-ray source, and an x-ray detector alignable with the x-ray source;
b. an image processing system operably connected to the gantry to control the operation of the x-ray source and x-ray detector to generate x-ray image data, the image processing system including a processing unit for processing the x-ray image data from the detector to form x-ray images, a database operably connected to the processing unit and storing instructions for operation of the processing unit, a display operably connected to the image processing system for presenting the x-ray images to a user, and a user interface operably connected to the image processing system to enable user input to the processing system; and
wherein the processing unit is configured to determine a position of at least two radiopaque markers located on an object within the anatomy of a patient, the at least two radiopaque markers identifying a proximal end of the object and a distal end of the object, to form one or more x-ray images of the object and the anatomy, and to present at least two different indicators representing the radiopaque markers within the one or more x-ray images on the display.

14. The x-ray system of claim 13, wherein the at least two different indicators comprise a first indicator having a first color and a second indicator having a second color.

15. The x-ray system of claim 13, wherein the one or more x-ray images comprise:
a. at least one 3D image of the object and the anatomy; and
b. a 2D image of the object and the anatomy.

16. The x-ray system of claim 13, wherein the one or more x-ray images comprise at least one 3D image of the object and the anatomy.

17. The x-ray imaging system of claim 13, wherein the x-ray imaging system is a C-arm system.

18. The x-ray imaging system of claim 17, wherein the x-ray imaging system is a CBCT system.

19. The x-ray imaging system of claim 13, wherein the object is a catheter.

20. The x-ray imaging system of claim 13, wherein the object is a stent.
